(19) United States
(12) Patent Application Publication
(10) Pub. No.: US 2023/0279410 A1
(43) Pub. Date: Sep. 7, 2023

(54) TOPICAL APPLICATION OF POLYNUCLEOTIDE MOLECULES FOR IMPROVING YIELD TRAITS OF PLANTS

(71) Applicants: IMI TAMI INSTITUTE FOR RESEARCH & DEVELOPMENT LTD., Haifa (IL); PLANTARC BIO LTD., Raanana (IL)

(72) Inventors: Dror SHALITIN, Raanana (IL); Noam GRIMBERG, Kibutz Lehavot Haviva (IL); ltay DODEK, Yokne'am Illit (IL)

(73) Assignee: PLANTARC BIO LTD., Raanana (IL)

(21) Appl. No.: 17/909,900

(22) PCT Filed: Mar. 15, 2021
(86) PCT No.: PCT/IL2021/050283

§ 371 Date: Sep. 7, 2022

Related U.S. Application Data
(60) Provisional application No. 62/990,309, filed on Mar. 16, 2020.
(60) Provisional application No. 63/063,683, filed on Aug. 10, 2020.
(60) Provisional application No. 63/063,696, filed on Aug. 10, 2020.

(51) Int. Cl. C12N 15/82; A01H 6/46; A01H 6/54; A01H 6/20

(52) U.S. Cl. CPC C12N 15/8206; C12N 15/8218; C12N 15/8261; A01H 6/4636; A01H 6/542; A01H 6/20

(57) ABSTRACT

A composition including: (i) a ds RNA molecule of at least 18 contiguous nucleotides that are essentially identical or essentially complementary to a plant gene or a transcript of said plant gene; and (ii) a transfer agent that conditions a surface of a plant to permeation by the ds RNA molecule into cells of the plant; wherein permeation of the ds RNA molecule into cells of the plant causes a transient reduction in the expression of the gene and wherein the transient reduction in the expression of the gene causes a change in a yield-associated trait of the plant.

### SEQUENCE LISTING

A sequence listing in electronic (ASCII text file) format is filed with this application and incorporated herein by reference. The name of the ASCII text file is “Sequence_listing_v2.txt”;

the file was created on May 9, 2023; the size of the file is 2,626,261 bytes.

### BACKGROUND OF THE INVENTION

According to the United Nations Food and Agricultural Organization (UN FAO), the world's population will exceed 9.6 billion people by the year 2050, which will require significant improvements in agriculture to meet growing food demands. At the same time, conservation of resources, reduction in the use of fertilizers, pesticides, and herbicides, environmental sustainability, are increasingly important factors in how food is grown. There is a need for improved agricultural plants and farming practices that will enable an increased plant production using fewer resources, more environmentally sustainable inputs.

Yield is affected by various factors, such as the number and size of the plant organs, plant architecture (for example, the number of branches), seed filling, seed number, drought resistance, shattering, flowering and number of tillers, etc.

Today, crop performance is optimized primarily via technologies directed towards the interplay between crop genotype (e.g., plant breeding, genetically modified (GM) crops) and its surrounding environment (e.g., fertilizer, synthetic herbicides, pesticides). While these paradigms have assisted in doubling global food production in the past fifty years, yield growth rates have stalled in many major crops, driving an urgent need for novel solutions to crop yield improvement. In addition to their long development and regulatory timelines, public fears of GM-crops and synthetic chemicals have challenged their use in many key crops and countries, resulting in a lack of acceptance for many GM traits and the exclusion of GM crops and many synthetic chemistries from some global markets. Thus, there is a significant need for innovative, effective, environmentally-sustainable, and publicly-acceptable approaches to improve the yield.

### SUMMARY OF THE INVENTION

Provided herein are compositions and methods that provide increased yield in plants by suppressing expression of a yield-associated gene in a plant, by topically providing to the plant a composition comprising a polynucleotide molecule capable of hybridizing with the yield-associated gene or gene transcript and a transfer agent that conditions a surface of the plant to permeation by the polynucleotide molecule into cells of the plant, thereby improving a yield-associated trait of the plant. Non-limiting examples of yield-associated trait of the plant include: increased branching in the plant, grain size, increased number of panicles, increased number of tillers, increased seed, increase in a size of siliques of the plant, increase in the filling of the seed, increased seed number, increased heading, improved drought resistance, decreased shattering, decreased abscise tissue formation, decreased petals in the plant, late/early flowering, shortened/prolonged flowering period, delayed senescence, increased oil contents, improved oil composition, starch content, starch composition, carbohydrate content, carbohydrate composition, increased protein contents, improved protein composition, and any combination thereof. Each possibility is a separate embodiment.

According to some embodiments, the penetration of the polynucleotide molecule may cause a transient reduction in the expression of the gene, cause a non-permanent spatial and temporal effect on the plant and does not result in nor require the exogenous polynucleotide's integration into a chromosome of the plant. This approach has several advantages. First it circumvents the need in GMO legislation. Moreover, it is more sophisticated and an efficient approach as it is dynamic and allows user-determined application according to real-time needs, timing of the trait improvement and/or environmental conditions that need addressing. As a non-limiting example, during drought a farmer may decide to transiently inhibit expression of genes/transcripts to improve water stress resilience and to discontinue the inhibition once weather changes. As another non-limiting example, the farmer may decide to transiently inhibit genes/transcripts associated with early flowering of the plant in the event of unexpected rain, temperature change or the like.

Advantageously, the technology is applicable for use in various crops, including but not limited to corn, rice, soybean, cotton, canola, oilseed rape, tomato, potato, etc. and especially to crops with complex genomes such as wheat, strawberries or fruit-trees.

According to some embodiments, the polynucleotide molecules are provided in compositions that can permeate or be absorbed into living plant tissue to initiate systemic gene inhibition or regulation. In certain embodiments of the invention, the polynucleotide molecules ultimately provide to a plant an RNA (e.g. a dsRNA) or RNA-like molecule that is capable of hybridizing under physiological conditions in a plant cell to RNA transcribed from a target endogenous gene in the plant cell, thereby effecting (silencing or suppressing) expression of the target gene.

According to some embodiments, the silencing/suppressing of the target gene may directly improve the yield-associated trait of the plant. Alternatively, the silencing/suppressing of the target gene may indirectly improve the yield-associated trait of the plant. For example, silencing or suppression of the target gene, may change (increase or decrease) expression of another gene that improves the yield associated trait of the plant.

As a further important practical advantage, the topical application of the composition comprising the exogenous polynucleotide and the transfer agent does not require that the exogenous polynucleotide be physically bound to a particle, such as in biolistic-mediated introduction of polynucleotides associated with gold or tungsten particles into internal portions of a plant, plant part, or plant cell.

According to some embodiments, the polynucleotide molecules target the mRNA of the plant gene. According to some embodiments, the polynucleotide molecules target the translated region of the mRNA. According to some embodiments, the polynucleotide molecules target the untranslated region of the mRNA.

According to some embodiments, there is provided a composition comprising: (i) a polynucleotide molecule that comprises at least 18 contiguous nucleotides that are essentially identical or essentially complementary to a plant gene or a transcript of the plant gene; and (ii) a transfer agent that conditions a surface of a plant to permeation by the polynucleotide molecule into cells of the plant; wherein permeation of the polynucleotide molecule into cells of the plant causes a transient reduction in the expression of the gene and wherein the transient reduction in the expression of the gene causes a change in a yield-associated trait of the plant.

According to some embodiments, the yield-associated trait of the plant is selected from the group consisting of: increased grain/seed size, increased grain number, increased number of panicles/siliques, increased number of tillers, increased branching, increased seed size, increased seed filling, increased seed number, increased heading, improved drought resistance, decreased shattering, decreased abscise tissue formation, late-flowering, early-flowering, increased shattering, increased abscise tissue formation, decreased petals in the plant, increased protein content of the plant, increased carbohydrate content of the plant, increased oil content of the plant, improved oil composition of the plant, starch content, starch composition, carbohydrate content, carbohydrate composition and any combination thereof. Each possibility is a separate embodiment.

According to some embodiments, there is provided a composition suitable for topical application on plants, composition comprising a dsRNA molecule that comprises at least 18 contiguous nucleotides that are essentially identical or essentially complementary to a portion of a plant gene or a transcript of the plant gene; and a transfer agent configured to facilitate permeation of the dsRNA molecule into cells of the plant, wherein permeation of the dsRNA molecule into cells of the plant causes a transient reduction in the expression of the gene.

The transient reduction in the expression of the gene causes a change in a trait of the plant.

The trait of the plant selected from the group consisting of increased branching, increased grain filling, increased trehalose-6-phosphate (T6P) levels, increased number of panicles, increased seed filling, increased seed number, increased seed size, decreased shattering, decreased abscise tissue formation, increased number of tillers, increased heading in the plant, petal reduction, increase siliques size, late or early flowering, delayed senescence and any combination thereof; or selected from the group consisting of increased branching, increased grain filling, increased number of panicles, increased seed filling, increased seed number, increased seed size, decreased shattering, decreased abscise tissue formation, increased number of tillers, increased heading in the plant, petal reduction, increase siliques size and any combination thereof; or selected from the group consisting of increased branching, increased grain filling, increased number of panicles, increased seed filling, increased seed number, decreased shattering, decreased abscise tissue formation, increased number of tillers, petal reduction, increase siliques size and any combination thereof. Each possibility is a separate embodiment.

According to some embodiments, the plant gene is selected from ADPG1, PTL, CKX2, BRC1, KIN1, SKIN1, PIN5b, JAG1, BS1, PLDα1 and/or or any homolog or combination thereof. Each possibility is a separate embodiment. According to some embodiments, the plant gene is selected from the composition of claim 1, wherein the plant gene is selected from ADPG1, PTL, CKX2, BRC1 and/or or any homolog or combination thereof. Each possibility is a separate embodiment.

According to some embodiments, the plant is an oilseed rape plant, and the dsRNA molecule a dsRNA molecule including at least 18 contiguous nucleotides that are essentially identical or essentially complementary to a portion of a sequence encoding any of the amino acid sequences set forth in NO SEQ ID NO: 599, SEQ ID NO: 650, SEQ ID NO: 522 and SEQ ID NO: 365. Each possibility is a separate embodiment. According to some embodiments, the dsRNA molecule has at least 80% homology to any of the sequences set forth in SEQ ID NO: 729, SEQ ID NO: 733, SEQ ID NO: 731 and SEQ ID NO: 730. Each possibility is a separate embodiment. According to some embodiments, the dsRNA molecule has at least 90% homology to any of the sequences set forth in SEQ ID NO: 729, SEQ ID NO: 733, SEQ ID NO: 731 and SEQ ID NO: 730. Each possibility is a separate embodiment.

According to some embodiments, the plant is a soybean plant, and the dsRNA molecule a dsRNA molecule including at least 18 contiguous nucleotides that are essentially identical or essentially complementary to a portion of a sequence encoding any of the amino acid sequences set forth in SEQ ID NO: 379, SEQ ID NO: 603, SEQ ID NO: 655, SEQ ID NO: 564, SEQ ID NO: 517, SEQ ID NO: 480 and SEQ ID NO: 488. Each possibility is a separate embodiment.

According to some embodiments, the plant is a soybean plant, and the dsRNA molecule a dsRNA molecule including at least 18 contiguous nucleotides that are essentially identical or essentially complementary to a portion of a sequence encoding any of the amino acid sequences set forth in SEQ ID NO: 379, SEQ ID NO: 517, SEQ ID NO: 480 and SEQ ID NO: 488. Each possibility is a separate embodiment. According to some embodiments, the dsRNA molecule has at least 80% homology to any of the sequences set forth in SEQ ID NO: 734-741. Each possibility is a separate embodiment. According to some embodiments, the dsRNA molecule has at least 90% homology to any of the sequences set forth in SEQ ID NO: 734-741. Each possibility is a separate embodiment.

According to some embodiments, the plant is a rice plant, and the dsRNA molecule a dsRNA molecule including at least 18 contiguous nucleotides that are essentially identical or essentially complementary to a portion of a sequence encoding any of the amino acid sequences set forth in SEQ ID NO: 407, SEQ ID NO: 610, SEQ ID NO: 659, SEQ ID NO: 589, SEQ ID NO: 416 and SEQ ID NO: 450. Each possibility is a separate embodiment.

According to some embodiments, the plant is a rice plant, and the dsRNA molecule a dsRNA molecule including at least 18 contiguous nucleotides that are essentially identical or essentially complementary to a portion of a sequence encoding any of the amino acid sequences set forth in SEQ ID NO: 407, SEQ ID NO: 416 and SEQ ID NO: 450. Each possibility is a separate embodiment. According to some embodiments, the dsRNA molecule has at least 80% homology to any of the sequences set forth in SEQ ID NO: 742-747. Each possibility is a separate embodiment. According to some embodiments, the dsRNA molecule has at least 90% homology to any of the sequences set forth in SEQ ID NO: 742-747. Each possibility is a separate embodiment.

According to some embodiments, the dsRNA molecule is at least about 50, bases in length. According to some embodiments, the dsRNA molecule is at least about 200, bases in length.

According to some embodiments, the transfer agent comprises N, N-dimethyl Decanamide, coco amidopropyldimethyamine, Siloxane Polyalkyleneoxide Copolymer, AG-RHO® EM-30, Dimethylamide of C8/C10 fatty acid, esterified copolymer of glycerol, trisiloxane ethoxylate or any combination thereof. Each possibility is a separate embodiment.

According to some embodiments, the transfer agent may be any of the transfer agents set forth in TABLE 1.

According to some embodiments, there is provided a method for topically applying to a plant surface the composition as essentially disclosed herein.

According to some embodiments, the applying comprises spraying the composition onto the surface of a plant. According to some embodiments, the composition is sprayed onto the surface of a plant with a boom that extends over a crop, a boomless sprayer, an agricultural sprayer, a crop-dusting airplane, a pressurized backpack sprayer, a track sprayer, or a laboratory sprayer/submerger. Each possibility is a separate embodiment.

According to some embodiments, the applying comprises providing the composition through an irrigation system.

According to some embodiments, the plant surface is the surface of one or more plant part selected from the group consisting of hypocotyl, cotyledon, leaf, flower, stem, tassel, meristem, pollen, ovule, and fruit. Each possibility is a separate embodiment.

According to some embodiments, the method further includes timing the applying of the composition at a desired developmental stage of the plant, as essentially explained herein for example in TABLE 2.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

FIG. 1 shows the contact angle (indicative of penetration) as a function of time after application of target agent mixture on a leaf of an oil seed rape plant;

FIG. 2 shows exemplary pictures of flower morphology of oilseed rape plants, here Brassica napus plants ectopically sprayed with 10 μg/ml of dsRNA set forth in SEQ ID NO: 733 targeting the sequence set forth in SEQ ID NO: 286. Control treated plants with normal petal morphology (left). BnPTL dsRNA treated plants causing flowers with changed petal morphology (right).

FIG. 3 shows exemplary pictures of oilseed rape plants, here Brassica napus plants ectopically sprayed with 10 μg/ml dsRNA set forth in SEQ ID NO: 730 targeting BnBRC1 and the sequence set forth in SEQ ID NO: 1 vs. control plants (Ctrl) plants. The total number of branches each group is depicted.

FIG. 4 shows average branch number per oilseed rape plants, here Brassica napus plants ectopically sprayed with 1 μg/ml or 10 μg/ml of dsRNA set forth in SEQ ID NO: 730 targeting the BnBRC1 sequence set forth in SEQ ID NO: 1 or plant sprayed with surfactant solution only. * indicates a significant change of treatment compared to the control plants (Ctrl) (P<0.1).

FIG. 5A shows the average seed weight per 0.8 m², of oilseed rape plants, here Brassica napus plants sprayed with dsRNA set forth in SEQ ID NO: 733 targeting the sequence set forth in SEQ ID NO: 286 obtained for the 1^{st} field. * indicates a significant change of treatment as compared to the control plants (Ctrl) (P<0.1).

FIG. 5B shows the average seed weight per 0.8 m², of oilseed rape plants, here Brassica napus plants sprayed with dsRNA set forth in SEQ ID NO: 733 targeting the sequence set forth in SEQ ID NO: 286 obtained for the 2^{nd} field. * indicates a significant change of treatment as compared to the control plants (Ctrl) (P<0.1).

FIG. 6A shows the average oil content percentage of oilseed rape plants, here Brassica napus plants sprayed with dsRNA set forth in SEQ ID NO: 733 targeting the sequence set forth in SEQ ID NO: 286 obtained for the 1^{st} field. * indicates a significant change of treatment as compared to the control plants (Ctrl) (P<0.1).

FIG. 6B shows the average oil content percentage of oilseed rape plants, here Brassica napus plants sprayed with dsRNA set forth in SEQ ID NO: 733 targeting the sequence set forth in SEQ ID NO: 286 obtained for the 2^{nd} field. * indicates a significant change of treatment as compared to the control plants (Ctrl) (P<0.1).

FIG. 7 shows average branch number per oilseed rape plants (Brassica napus) plants ectopically sprayed with 1 μg/ml or 10 μg/ml of dsRNA set forth in SEQ ID NO: 730 targeting the sequence set forth in SEQ ID NO: 1 or plant sprayed with surfactant solution only. * indicates a significant change of treatment compared to the control plants (Ctrl) (P<0.1).

FIG. 8A shows the average seed weight per 0.8 m², of oilseed rape plants (Brassica napus) plants sprayed with dsRNA set forth in SEQ ID NO: 731 targeting the sequence set forth in SEQ ID NO: 158 obtained for the 1^{st} field. * indicates a significant change of treatment as compared to the control plants (Ctrl) (P<0.1).

FIG. 8B shows the average seed weight per 0.8 m², of oilseed rape plants (Brassica napus) plants sprayed with dsRNA set forth in SEQ ID NO: 731 targeting the sequence set forth in SEQ ID NO: 158 obtained for the 2^{nd} field. * indicates a significant change of treatment as compared to the control plants (Ctrl) (P<0.1).

FIG. 9A shows the average seed weight per 0.8 m², of oilseed rape plants (Brassica napus) plants sprayed with dsRNA set forth in SEQ ID NO: 729 targeting the sequence set forth in SEQ ID NO: 235 obtained for the 1^{st} field. * indicates a significant change of treatment as compared to the control plants (Ctrl) (P<0.1).

FIG. 9B shows the average seed weight per 0.8 m², of oilseed rape plants (Brassica napus) plants sprayed with dsRNA set forth in SEQ ID NO: 729 targeting the sequence set forth in SEQ ID NO: 235 obtained for the 2^{nd} field. * indicates a significant change of treatment as compared to the control plants (Ctrl) (P<0.1).

FIG. 10A shows the average oil content percentage of oilseed rape plants (Brassica napus) plants sprayed with dsRNA set forth in SEQ ID NO: 729 targeting the sequence set forth in SEQ ID NO: 235 obtained for the 1^{st} field. * indicates a significant change of treatment as compared to the control plants (Ctrl) (P<0.1).

FIG. 10B shows the average oil content percentage of oilseed rape plants (Brassica napus) plants sprayed with dsRNA set forth in SEQ ID NO: 729 targeting the sequence set forth in SEQ ID NO: 235 obtained for the 2^{nd} field. * indicates a significant change of treatment as compared to the control plants (Ctrl) (P<0.1).

FIG. 11 shows the average number of tillers per rice plant (Oryza sativa) treated with 1 μg/ml or 10 μg/ml of dsRNA set forth in SEQ ID NO: 742 targeting the sequence set forth in SEQ ID NO: 43 or plant sprayed with surfactant solution only. * indicates a significant change of treatment compared to the control plants (Ctrl) (P<0.1).

FIG. 12 shows the average number of branches soybean plant (Glycine max) treated with 1 μg/ml or 10 μg/ml of −dsRNA set forth in SEQ ID NO: 734 targeting the sequence set forth in SEQ ID NO: 15 or plant sprayed with surfactant solution only. * indicates a significant change of treatment compared to the control plants (Ctrl) (P<0.1).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The following definitions and methods are provided to better define the present invention and to guide those of ordinary skill in the art in the practice of the present invention. Unless otherwise noted, terms are to be understood according to conventional usage by those of ordinary skill in the relevant art.

Where a term is provided in the singular, the inventors also contemplate aspects of the invention described by the plural of that term.

As used herein, the terms “polynucleotide molecule” and “polynucleotide” may be used interchangeably and refer to any polynucleotide composed of 18 or more nucleotides covalently bonded in a chain and capable of hybridizing to DNA and RNA molecules under physiological conditions. According to some embodiments, the polynucleotide may be a synthetic and/or artificial polynucleotide molecule. According to some embodiments, the polynucleotide molecule is a biopolymer. According to some embodiments, the biopolymer is a DNA (deoxyribonucleic acid) or an RNA (ribonucleic acid) molecule.

According to some embodiments, the polynucleotide molecules target the mRNA of the plant gene. According to some embodiments, the polynucleotide molecules target the translated region of the mRNA. According to some embodiments, the polynucleotide molecules target the untranslated region (UTR) of the mRNA.

As used herein, the terms “DNA”, “DNA molecule” and “DNA polynucleotide molecule” refer to a single-stranded DNA or double-stranded DNA molecule of genomic or synthetic origin, such as, a polymer of deoxyribonucleotide bases or a DNA polynucleotide molecule.

As used herein, the terms “DNA sequence”, “DNA nucleotide sequence”, and “DNA polynucleotide sequence” refer to the nucleotide sequence of a DNA molecule.

As used herein, the term “gene” refers to any portion of a nucleic acid that provides for expression of a transcript or encodes a transcript. A “gene” thus includes, but is not limited to, a promoter region, 5′ untranslated regions, transcript encoding regions that can include intronic regions, and 3′ untranslated regions.

As used herein, the terms “RNA”, “RNA molecule”, and “RNA polynucleotide molecule” refer to a single-stranded RNA or double-stranded RNA molecule of genomic or synthetic origin, such as, a polymer of ribonucleotide bases that comprise single or double stranded regions or any other structural elements.

Unless otherwise stated, nucleotide sequences in the text of this specification are given, when read from left to right, in the 5′ to 3′ direction. The nomenclature used herein is that required by Title 37 of the United States Code of Federal Regulations § 1.822 and set forth in the tables in WIPO Standard ST.25 (1998), Appendix 2, Tables 1 and 3.

As used herein, a “plant surface” refers to any exterior portion of a plant. Plant surfaces thus include, but are not limited to, the surfaces of flowers, stems, tubers, fruit, anthers, pollen, leaves, roots, or seeds. A plant surface can be on a portion of a plant that is attached to other portions of a plant or on a portion of a plant that is detached from the plant.

As used herein, the phrase “polynucleotide is not operably linked to a promoter” refers to a polynucleotide that is not covalently linked to a polynucleotide promoter sequence that is specifically recognized by either a DNA dependent RNA polymerase II protein or by a viral RNA dependent RNA polymerase in such a manner that the polynucleotide will be transcribed by the DNA dependent RNA polymerase protein or viral RNA dependent RNA polymerase. A polynucleotide that is not operably linked to a promoter can be transcribed by a plant RNA dependent RNA polymerase.

As used herein, SEQ ID NO: 1-364 and 729-747, though displayed in the Sequence Listing in the form of ssDNA, encompass dsDNA equivalents, dsRNA equivalents, ssRNA equivalents, ssRNA complements, ssDNA as shown, and ssDNA complements.

As used herein, the term “transfer agent” may refer to any agent rendering the plant prone to receiving polynucleotides when the agent is applied to the surface of the plant. According to some embodiments, a transfer agent is an agent that conditions the surface of plant tissue, e.g., seeds, leaves, stems, roots, flowers, or fruits, to permeation by the polynucleotide molecules into plant cells. Chemical agents for conditioning or transfer include (a) wetting agents, (a) surfactants, (b) an organic solvent or an aqueous solution or aqueous mixtures of organic solvents, (c) oxidizing agents, (d) acids, (e) bases, (f) oils, (g) enzymes, or combinations thereof.

According to some embodiments the transfer agent may be selected from N, N-dimethyl Decanamide, coco amidopropyldimethyamine, Siloxane Polyalkyleneoxide Copolymer, AG-RHO® EM-30, Dimethylamide of C8/C10 fatty acid, esterified copolymer of glycerol, trisiloxane ethoxylate or any combination thereof.

A non-limiting example of a suitable transfer agent include organosilicone compounds.

As used herein, the phrase “organosilicone preparation” refers to a liquid comprising one or more organosilicone compounds, wherein the liquid or components contained therein, when combined with a polynucleotide in a composition that is topically applied to a target plant surface, better enables the polynucleotide to enter a plant cell. Exemplary organosilicone preparations include, but are not limited to, preparations marketed under the trade names Silwet® or BREAKTHRU®. In certain embodiments, an organosilicone preparation can better enable a polynucleotide to enter a plant cell in a manner permitting a polynucleotide mediated suppression of target gene expression in the plant cell.

A non-limiting example of a specific suitable transfer agent includes Silwet® L-77, which is a modified trisiloxane that combines a very low molecular weight trisiloxane with a polyether group. It is characterized by remarkable interfacial activity, which can result in dramatically reduced aqueous surface tension, outstanding spreading or levelling and stabilized foam. All of which may be achieved using a fraction of the typical concentration levels of organic or fluorocarbon surfactants.

Another non-limiting example of a specific suitable transfer agent includes GENAGEN™ 4166 (Clariant, Material no.: 10783626892). GENAGEN™ 4166 is a dimethylamide based on naturally derived fatty acids.

Another non-limiting example of a specific suitable transfer agent includes SYNERGEN® GL 5: (Clarian, Material no.: 20072326894). SYNERGEN® GL 5 is a polyglycerol ester-based adjuvant, which is a TAE-free surfactant derived from renewable resources. Another non-limiting example of a specific suitable transfer agent includes GENAGEN™ 4296 (Clariant, Material no.: 10783926892). GENAGEN™ 4296 is a dimethylamide based on naturally derived fatty acids.

Another non-limiting example of a specific suitable transfer agent includes SYNERGEN® GA: (Clariant, Material no.: 27251626894). SYNERGEN® GA is a novel biological enhancer of salts of agrochemicals, based on alkylglucamines. It is a sugar-based surfactant with a renewable carbon index (RCI) above 95% and therefore with an excellent ecological profile.

Another non-limiting example of a specific suitable transfer agent includes GENAGEN™ SC 35 (Clariant, Material no.: 25923226892). GENAGEN™ SC 35 is a basic surfactant blend of alkyl diglycol ether sulfate sodium salt and coconut fatty acid monoethanolamide.

Another non-limiting example of a specific suitable transfer agent includes HOSTAPHAT® 1306 (Clariant, Material no.: 13326826900). HOSTAPHAT® 1306 is an anionic emulsifier used for the emulsion polymerization of monomers like pure acrylic, styrene-acrylic acid esters and vinyl acetate.

Another non-limiting example of a specific suitable transfer agent includes SURFECO PLUS™ (Latro). SURFECO PLUS™ is silicone-based adjuvant used for the modify to physical properties and enhancing biological activities of agrochemicals.

Additional suitable transfer agents and their chemical properties are summarized in TABLE 1 below.

TABLE 1
Transfer agents

| Material | Chemistry | CAS | Manufacturer |
|---|---|---|---|
| Adsee ® C80W | cocoamidopropyl- | 68140-01-2 | Nouryon |
| | dimethyamine | | |
| AG-RHO ® | — | — | Solvay |
| EM-30 | | | |
| Silwet ® L-77 | Siloxane | 27306-78-1 & | Momentive |
| AG | Polyalkyleneoxide | 67674-67-3 | |
| | Copolymer | | |
| HALLCOMID ® | N,N-dimethyl | 14433-76-2 | Stepan |
| M-10 | Decanamide | | |
| GENAGEN ™ | Dimethylamide | 10783626892 | Clariant |
| 4166 | of C8/C10 fatty acid | | |
| SYNERGEN ® | Copolymer of | 20072326894 | Clariant |
| GL5 | glycerol, esterified | | |
| SURFECO | trisiloxane | | Latro |
| PLUS ™ | ethoxylate | | |
| BREAK- | — | — | Evonik |
| THRU ® S301 | | | |
| BREAK- | — | — | Evonik |
| THRU ® S278 | | | |
| BREAK- | — | — | Evonik |
| THRU ® S233 | | | |
| TEGOPREN ® | — | — | Evonik |
| 5873 | | | |

As used herein, the phrases “improved yield” refers to any measurable improvement in yield. In certain embodiments, an improvement in yield in a plant or plant part can be determined in a comparison to a control plant or plant part that has not been treated with a composition comprising a polynucleotide. When used in this context, a control plant is a plant that has not undergone treatment with polynucleotide and a transfer agent. Such control plants would include, but are not limited to, untreated plants or mock treated plants.

Non-limiting examples of traits affected by the herein disclosed compositions include: increased branching, increased seed filing, increased seed number, improved drought resistance, decreased shattering, decreased abscise tissue formation, late/early flowering and any combination thereof. Each possibility is a separate embodiment.

Non-limiting examples of rice plant traits affected by the herein disclosed compositions include: increased grain size, increased number of panicles, increased number of tillers, increased heading and any combination thereof. Each possibility is a separate embodiment.

Non-limiting examples of oilseed rape traits affected by the herein disclosed compositions include: increased grain size, increased number of panicles, increased number of tillers, increased branching, increased seed filling increased seed number, increased heading, improved drought resistance, decreased shattering, decreased abscise tissue formation, late/early flowering, shorten/prolonged flowering period and any combination thereof. Each possibility is a separate embodiment.

According to some embodiments, the plant may be any cultivated plant, such as, but not limited to, oilseed rape, rapeseed, rice, wheat, barley, soy, peanut, cotton, corn, sorghum, sugarcane, sugar beet, beans, sunflower potato, sweet potato, alfalfa, banana, apricot, grape, apple, peach, prune, citrus, date, palm oil, pepper, tomato, broccoli, onion, melon, watermelon, yam, cassava. Each possibility is a separate embodiment.

According to some embodiments, the plant may be a soy plant, a rice plant or an oilseed rape plant. Each possibility is separate embodiment. According to some embodiments, the soybean plant may be of the species Glycine max. According to some embodiments, the rice plant may be of the species Oryza sativa. According to some embodiments, the oilseed rape plant may be of the species Brassica napus. Each possibility is separate embodiment.

According to some embodiments, the gene targeted by the polynucleotide molecule may be referred to by a scientific name as used in one species, e.g. in Arabidopsis thaliana. However, it is understood to one of ordinary skill in the art that aliases as well as homologs of another species referred to by another name (alias/homolog) are encompassed within the stated scientific name. As a non-liming example, when the gene targeted by the polynucleotide molecule is referred to as BRC1, it encompasses the aliases/homologs TB1/FC1.

According to some embodiments, the target gene may have a nucleotide sequence selected from any of the nucleotide sequences set forth in SEQ ID NO: 1-364. Each possibility is a separate embodiment. According to some embodiments, the target gene may encode an amino acid sequence selected from any of the amino acid sequences set forth in SEQ ID NO: 365-728. Each possibility is a separate embodiment. According to some embodiments, the polynucleotide (i.e. the dsRNA) may have the nucleotide sequence set forth in SEQ ID NO: 729-747. Each possibility is a separate embodiment.

According to some embodiments, the polynucleotide molecule is dsRNA having a polynucleotide sequence essentially identical to the sequence set forth in any one of SEQ ID NO: 729-747 or major part thereof. As used herein, the term “major part thereof” with referral to the dsRNA refers the dsRNA being at least 80% identical to at least 18-20 contiguous base pairs of the sequence set forth in any one of SEQ ID NO: 729-747, the dsRNA being at least 85% identical to at least 18-20 contiguous base pairs of the sequence set forth in any one of SEQ ID NO: 729-747, the dsRNA being at least 90% identical to at least 18-20 contiguous base pairs of the sequence set forth in any one of SEQ ID NO: 729-747, the dsRNA being at least 95% identical to at least 18-20 contiguous base pairs of the sequence set forth in any one of SEQ ID NO: 729-747, or the dsRNA being at least 98% identical to at least 18-20 contiguous base pairs of the sequence set forth in any one of SEQ ID NO: 729-747. Each possibility is a separate embodiment.

As used herein, the term “essentially identical to” with referral to the dsRNA refers to a dsRNA sequence having at least 80%, at least 90%, at least 95% homology, or at least 98% homology to a portion of the nucleotide sequence set forth in SEQ ID NO: 1-364. As used herein, the term “a portion of the nucleotide sequence set forth” refers to a portion of the nucleotide sequence target by the dsRNA having an essentially same length as the dsRNA. As a non-limiting example, if the dsRNA has a length of 18 bp, the portion of the nucleotide sequence targeted by the dsRNA has a length of about 18 bp. As another non-limiting example, if the dsRNA has a length of 200 bp, the portion of the nucleotide sequence targeted by the dsRNA has a length of about 200 bp.

As used herein, the terms “approximately” and “about” refer to +/−10%, or +/−5%, or +−2% vis-à-vis the range to which it refers. Each possibility is a separate embodiment.

According to some embodiments, the dsRNA targets BnADPG1 of oilseed rape plants (SEQ ID NO: 235) and has the polynucleotide sequence set forth in SEQ ID NO: 729.

According to some embodiments, the dsRNA targets BnBRC1 of oilseed rape plants (SEQ ID NO: 1) and has the polynucleotide sequence set forth in SEQ ID NO: 730.

According to some embodiments, the dsRNA targets BnCKX2 of oilseed rape plants (SEQ ID NO:158) and has the polynucleotide sequence set forth in SEQ ID NO: 731.

According to some embodiments, the dsRNA targets BnKIN10 of oilseed rape plants (SEQ ID NO: 62) and has the polynucleotide sequence set forth in SEQ ID NO: 732.

According to some embodiments, the dsRNA targets BnPTL of oilseed rape plants (SEQ ID NO: 286) and has the polynucleotide sequence set forth in SEQ ID NO: 733.

According to some embodiments, the dsRNA targets GmBRC1 of the soybean plant (SEQ ID NO: 15) and has the polynucleotide sequence set forth in SEQ ID NO: 734 or 735.

According to some embodiments, the dsRNA targets GmBS1 of the soybean plant (SEQ ID NO: 116) and has the polynucleotide sequence set forth in SEQ ID NO: 736 or 737.

According to some embodiments, the dsRNA targets GmJAG1 of the soybean plant (SEQ ID NO: 153) and has the polynucleotide sequence set forth in SEQ ID NO: 738 or 739.

According to some embodiments, the dsRNA targets GmPLDa1 of the soybean plant (SEQ ID NO: 124) and has the polynucleotide sequence set forth in SEQ ID NO: 740 or 741.

According to some embodiments, the dsRNA targets OsBRC1 of the rice plant (SEQ ID NO: 43) and has the polynucleotide sequence set forth in SEQ ID NO: 742 or 743.

According to some embodiments, the dsRNA targets OsPIN5b of the rice plant (SEQ ID NO: 86) and has the polynucleotide sequence set forth in SEQ ID NO: 744 or 745.

According to some embodiments, the dsRNA targets OsSKIN1 of the rice plant (SEQ ID NO: 52) and has the polynucleotide sequence set forth in SEQ ID NO: 746 or 747.

According to some embodiments, the composition and method for applying same may include the timing of the applying of the composition to a desired developmental trait of the plant. As a non-limiting example, the applying of a composition comprising a polynucleotide configured to target the gene BS1 (or other gene involved in the regulation of seed filling) may be timed to when the plant is at seed filling stage (R3-R5 dev. stage). As another non-limiting example, the applying of a composition comprising a polynucleotide configured to target the gene BRC1 (or other gene the reduced expression of which causing increased branching or number of tillers) may be timed to the bolting stage (R1-R2 dev. stage) in soy plants or to the vegetative phase in the development of axillary buds in rice plants. As another non-limiting example, the applying of a composition comprising a polynucleotide configured to target the gene JAG1 (or other gene increases the number of seeds) may be timed to when the plant is at flowering stage (R1-R2 dev. stage). As another non-limiting example, the applying of a composition comprising a polynucleotide configured to target the gene SGR1 (or other target gene the reduction of which increased resistance to drought) may be timed to a period of unexpected drought. As another non-limiting example, the applying of a composition comprising a polynucleotide configured to target the gene AGL1 (or other target gene the reduction of which causes reduced shattering) may be timed to when the siliques of the plant mature. As another non-limiting example, the applying of a composition comprising a polynucleotide configured to target the gene FT5a (or other target gene the reduction of which is involved in the regulation of flowering) may be timed to when the plant is at the flowering stage. As another non-limiting example, the applying of a composition comprising a polynucleotide configured to target the gene GNI1 (or other gene that regulates grain size) may be timed to when plant is at seed filling stage.

According to some embodiments, the composition may include more than one polynucleotide sequence (different sequences), such as 2, 3, 4, 5 or more polynucleotide sequences. Each possibility is a separate embodiment.

According to some embodiments, the two or more polynucleotide sequences may target the same target gene. i.e. they may be directed to different part of the sequence of the same target gene.

According to some embodiments, the two or more polynucleotide sequences may target different target genes.

According to some embodiments, the different target genes may be directed to a same yield associated trait (e.g., decreased shattering). As a non-limiting example, the two or more polynucleotide sequences may target AGL1 and PDH1. As another non-limiting example, the two or more polynucleotide sequences may target two or more of JAG1, JAG2, CKX1, OTU1 all affecting seed number.

According to some embodiments, the different target genes may be directed to different yield associated traits (e.g., increased drought resistance and seed filling). As a non-limiting example, a first of the two or more polynucleotide sequences may target ERA1, SGR1, SGR2, ACO2, CER9 or CytG, whereas a second of the two or more polynucleotide sequences may target BS1, PLD, ACO3 or PDHK.

As used herein, the phrase “reduction in the expression”, when used in the context of a transcript or a protein in a plant or plant part, refers to any measurable decrease in the level of transcript or protein in a plant or plant part. In certain embodiments, a reduction of the level of a transcript or protein in a plant or plant part can be determined in a comparison to a control plant or plant part that has not been treated with a composition comprising a polynucleotide and a transfer agent.

As used herein, the phrase “wherein said plant does not comprise a transgene” refers to a plant that lacks either a DNA molecule comprising a promoter that is operably linked to a polynucleotide or a recombinant viral vector.

As used herein, the term “transgene” describes a segment of DNA containing a gene sequence isolated from one organism and is introduced into the DNA of a different organism.

As used herein, the phrase “suppressing expression” or “reducing expression”, when used in the context of a gene, refers to any measurable decrease in the amount and/or activity of a product encoded by the gene. Thus, expression of a gene can be suppressed when there is a reduction in levels of a transcript from the gene, a reduction in levels of a protein encoded by the gene, a reduction in the activity of the transcript from the gene, a reduction in the activity of a protein encoded by the gene, any one of the preceding conditions, or any combination of the preceding conditions. In this context, the activity of a transcript includes, but is not limited to, its ability to be translated into a protein and/or to exert any RNA-mediated biologic or biochemical effect. In this context, the activity of a protein includes, but is not limited to, its ability to exert any protein-mediated biologic or biochemical effect. When used in this context, a control plant or plant part is a plant or plant part that has not undergone treatment with polynucleotide and a transfer agent.

As used herein, the term “transient” when used in the context of a reduction/suppression in the expression of a gene, refers to a time-limited reduction in the expression of the gene that last only as long as the polynucleotide permeated into the cell is not degraded, as opposed to long-term expression typically referred to as “table expression”.

As used herein, the term “transcript” corresponds to any RNA that is produced from a gene by the process of transcription. A transcript of a gene can thus comprise a primary transcription product which can contain introns or can comprise a mature RNA that lacks introns.

As used herein, the term “homolog”, with reference to polynucleotide molecule, refers to a degree of sequence identity or similarity (homology) between nucleotide sequences indicative of shared ancestry. Two segments of DNA can have shared ancestry because of either a speciation event (orthologs) or a duplication event (paralogs). According to some embodiments a homolog may refer to a polynucleotide having substantially from about 70% to about 99% sequence identity, or more preferably from about 80% to about 99% sequence identity, or most preferable from about 90% to about 99% sequence identity, to about 99% sequence identity, to the referent nucleotide sequences of a referent polynucleotide molecule. Each possibility is a separate embodiment.

As used herein, the term “sequence identity”, “sequence similarity” or “homology” is used to describe sequence relationships between two or more nucleotide sequences. The percentage of “sequence identity” between two sequences is determined by comparing two optimally aligned sequences. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

According to some embodiments, the composition may further include a carrier. According to some embodiments the carrier may be a liquid.

As used herein, the term “liquid” refers to both homogeneous mixtures such as solutions and non-homogeneous mixtures such as suspensions, colloids, micelles, and emulsions. Each possibility is a separate embodiment.

According to some embodiments, the liquid may be an aqueous solution. According to some embodiments, the liquid may be an oil or an oil mixture.

According to some embodiments, the polynucleotide may be naked. As used herein the term “naked” refers to the polynucleotide being non-encapsulated. The naked polynucleotide may however be modified and/or conjugated.

According to other embodiments, the polynucleotide may be encapsulated.

According to some embodiments, the polynucleotide may be delivered via and/or enclosed in a vehicle such as but not limited to a nanoparticle, a liposome, a micelle or the like.

Provided herein are certain methods and polynucleotide compositions that can be applied to living plant cells/tissues to suppress expression of target genes and that provide improved yield to a plant in need of the benefit. Also provided herein are plants and plant parts exhibiting improved yield as well as processed products of such plants or plant parts. The compositions may be topically applied to the surface of a plant, such as to the surface of a leaf. The composition can be applied to various plants including, but not limited to plants of the Brassicaceae family, the Fabaceae family or the Poaceae family of plants, such as but not limited to soybean plants, rice plants, oilseed rape plants and/or rapeseed plants. Each possibility is a separate embodiment.

As used herein, “polynucleotide” refers to a DNA or RNA molecule containing multiple nucleotides and generally refers both to “oligonucleotides” (a polynucleotide molecule of 18-25 nucleotides in length) and longer polynucleotides of 26 or more nucleotides. Embodiments of this invention include compositions including polynucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e. g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

Polynucleotide compositions used in the various embodiments of this invention include compositions including polynucleotides, including: RNA or DNA or RNA/DNA hybrids or chemically modified polynucleotides or artificial polynucleotides or a mixture thereof. In certain embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In certain embodiments, the polynucleotide includes non-canonical nucleotides such as inosine, thiouridine, or pseudouridine. In certain embodiments, the polynucleotide includes chemically modified nucleotides. Examples of chemically modified oligonucleotides or polynucleotides are well known in the art. Illustrative examples include, but are not limited to, the naturally occurring phosphodiester backbone of an polynucleotide which can be partially or completely modified with phosphorothioate, phosphorodithioate, or methylphosphonate internucleotide linkage modifications, modified nucleoside bases or modified sugars can be used in polynucleotide synthesis, and polynucleotides can be labeled with a fluorescent moiety (e. g., fluorescein or rhodamine) or other label (e. g., biotin).

According to some embodiments, the dsRNA may be chemically modified on one or both strands to improve stability, expand the half-life of the dsRNA in-vivo, increase the bio-distribution and pharmacokinetic properties of the dsRNA, target the dsRNA to specific cells, increase the target binding affinity, and/or improve drug delivery. As a non-limiting example, the dsRNA may be modified to include methyl-groups to the 2′ position of the ribosyl ring of the 2nd base of the dsRNA. As another non-limiting example, the dsRNA may be modified to include a 3′ overhang.

According to some embodiments, the modifications can be included in the dsRNA. According to some embodiments, the modification does not prevent the dsRNA composition from serving as a substrate for Dicer. In one embodiment, one or more modifications are made that enhance Dicer processing of the dsRNA. In a second embodiment, one or more modifications are made that result in more effective RNAi generation. In a third embodiment, one or more modifications are made that support a greater RNAi effect. In a fourth embodiment, one or more modifications are made that result in greater potency per each dsRNA molecule to be delivered to the cell. Modifications can be incorporated in the 3′-terminal region, the 5′-terminal region, in both the 3′-terminal and 5′-terminal region or in some instances in various positions within the sequence. With the restrictions noted above in mind, any number and combination of modifications can be incorporated into the dsRNA. Where multiple modifications are present, they may be the same or different. Modifications to bases, sugar moieties, the phosphate backbone, and their combinations are contemplated. Either 5′-terminus can be phosphorylated.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied.

Polynucleotides can be single- or double-stranded RNA, single- or double-stranded RNA, with structural features, single- or double-stranded DNA, double-stranded DNA/RNA hybrids, and modified analogues thereof. In certain embodiments of the invention, the polynucleotides that provide single-stranded RNA in the plant cell may be: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, (i) a double-stranded, hybridized RNA/DNA molecule, and (j) single-stranded RNA molecule (ssRNA) that self-hybridizes to form a structural motifs (such as stem-loop) or combinations thereof. In certain embodiments, these polynucleotides can comprise both ribonucleic acid residues and deoxyribonucleic acid residues. In certain embodiments, these polynucleotides include chemically modified nucleotides or non-canonical nucleotides. In certain embodiments of the methods, the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In certain embodiments where the polynucleotide is a dsRNA, the anti-sense strand will comprise at least 18 nucleotides that are essentially complementary to the target gene. In certain embodiments the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression.

The polynucleotide molecules of the present invention are designed to modulate expression by inducing regulation or suppression of an endogenous target gene in a plant and are designed to have a nucleotide sequence essentially identical or essentially complementary to the nucleotide sequence of an endogenous target gene of a plant or to the sequence of RNA transcribed from an endogenous target gene of a plant, which can be coding sequence or non-coding sequence.

By “essentially identical” or “essentially complementary” it is meant that the polynucleotides (or at least one strand of a double-stranded polynucleotide) have sufficient identity or complementarity to the endogenous gene or to the RNA transcribed from the endogenous target gene (e.g., the transcript) to suppress expression of the endogenous target gene (e.g. to effect a reduction in levels or activity of the gene transcript and/or encoded protein.

Polynucleotides of the methods and compositions provided herein need not have 100 percent identity to a complementarity to the endogenous target gene or to the RNA transcribed from the endogenous target gene (i.e. the transcript) to suppress expression of the endogenous target gene (i.e. to effect a reduction in levels or activity of the gene transcript or encoded protein). Thus, in certain embodiments, the polynucleotide or a portion thereof is designed to be essentially identical to, or essentially complementary to, a sequence of at least 18 or 19 contiguous nucleotides in either the target gene or messenger RNA transcribed from the target gene (e.g., the transcript). In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene (e.g., the transcript). In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

In certain embodiments, polynucleotides used in the methods and compositions provided herein can be essentially identical or essentially complementary to any of: i) conserved regions of target genes of both monocot and dicot plants; ii) conserved regions of target genes of monocot plants; or iii) conserved regions of target genes of dicot plants. Such polynucleotides that are essentially identical or essentially complementary to such conserved regions can be used to improve delayed senescence and/or improved yield by suppressing expression of target genes in various dicot.

Polynucleotides containing mismatches to the target gene or transcript can thus be used in certain embodiments of the compositions and methods provided herein. In certain embodiments, a polynucleotide of 19 continuous nucleotides that is essentially identical or essentially complementary to the endogenous target gene or to an RNA transcribed from the target gene (e.g., the transcript) can have 1 or 2 mismatches to the target gene or transcript. In certain embodiments, a polynucleotide of 20 or more nucleotides that contains a contiguous 19 nucleotide span of identity or complementarity to the endogenous target gene or to an RNA transcribed from the target gene can have 1 or 2 mismatches to the target gene or transcript. In certain embodiments, a polynucleotide of 21 continuous nucleotides that is essentially identical or essentially complementary to the endogenous target gene or to an RNA transcribed from the target gene (e.g., the transcript) can have 1, 2, or 3 mismatches to the target gene or transcript. In certain embodiments, a polynucleotide of 22 or more nucleotides that contains a contiguous 21 nucleotide span of identity or complementarity to the endogenous target gene or to an RNA transcribed from the target gene can have 1, 2, or 3 mismatches to the target gene or transcript. In designing polynucleotides with mismatches to an endogenous target gene or to an RNA transcribed from the target gene, mismatches of certain types and at certain positions that are more likely to be tolerated can be used.

According to some embodiments, the target genes may be any of the target genes set forth in SEQ ID NO: 1-364) as well as orthologous target genes obtainable from other crops.

According to some embodiments, the plant may be an oilseed rape plant and the polynucleotide may reduce the level of a target gene having any of the nucleotide sequences set forth in SEQ ID NO: 1-14, 49-51, 62-66, 79-84, 104-115, 149-151, 158-199, 235-238, 252-258, 275-279, 286-290, 299-303, 311-318, 332-341 and 360-361 or major parts thereof. Each possibility is a separate embodiment.

According to some embodiments, the plant may be an oilseed rape plant and the polynucleotide may reduce the level of the protein having any of the amino acid sequences set forth in SEQ ID NO: 365-378, 413-415, 426-430, 443-448, 468-479, 513-515, 522-563, 599-602, 616-622, 639-643, 650-654, 663-667, 675-682, 696-705 and 724-725 or major parts thereof. Each possibility is a separate embodiment.

According to some embodiments, the plant may be an oilseed rape plant and the polynucleotide may reduce the level of the protein having any of the amino acid sequences set forth in SEQ ID NO: 1, 2, 9, 49, 62, 79, 80, 104, 149, 150, 158, 235, 252, 275, 276, 286, 299, 311, 332, 333 and 360 or major parts thereof. Each possibility is a separate embodiment.

According to some embodiments, the plant may be an oilseed rape plant and the polynucleotide may reduce the level of the protein having any of the amino acid sequences set forth in SEQ ID NO: 1, 2, 9, 49, 62, 79, 80, 104, 149, 150, 158, 235, 252 or major parts thereof. Each possibility is a separate embodiment.

According to some embodiments, the plant may be an oilseed rape plant and the polynucleotide may have the nucleotide sequence set forth in SEQ ID NO: 729-733.

According to some embodiments, the plant may be a soybean plant and the polynucleotide may reduce the level of a target gene having any of the nucleotide sequences set forth in SEQ ID NO: 15-42, 67-72, 116-148, 152-157, 200-224, 239-245, 291-294, 304-310, 319-325, 342-351 and 362 or any major part thereof. Each possibility is a separate embodiment.

According to some embodiments, the plant may be a soybean plant and the polynucleotide may reduce the level of a target gene having any of the nucleotide sequences set forth in SEQ ID NO: 15, 16, 22-31, 67-69, 116-124, 152-155, 200-204, 239, 240, 291-293, 304, 305, 319, 320, 342, 343, 345 and 362 or any major part thereof. Each possibility is a separate embodiment.

According to some embodiments, the plant may be a soybean plant and the polynucleotide may reduce the level of a target gene having any of the nucleotide sequences set forth in SEQ ID NO: 15, 16, 67-69, 116-124, 152-155, 200-204, 239, 240 or any major part thereof. Each possibility is a separate embodiment.

According to some embodiments, the plant may be a soybean plant and the polynucleotide may reduce the level of the protein having any of the amino acid sequences set forth in SEQ ID NO: 379-406, 431-436, 480-512, 516-521, 564-588, 603-609, 655-658, 668-674, 683-689, 706-715 and 726 or any major part thereof. Each possibility is a separate embodiment.

According to some embodiments, the plant may be a soybean plant and the polynucleotide may have the nucleotide sequences set forth in SEQ ID NO: 734-741.

According to some embodiments, the plant may be a rice plant and the polynucleotide may reduce the level of a target gene having any of the nucleotide sequences set forth in SEQ ID NO: 43-48, 52-61, 73-78, 85-103, 225-234, 246-251, 259-274, 280-285, 295-298, 326-331, 352-359, 363-364 or any major part thereof. Each possibility is a separate embodiment.

According to some embodiments, the plant may be a rice plant and the polynucleotide may reduce the level of a target gene having any of the nucleotide sequences set forth in SEQ ID NO: 43, 44, 52-57, 73-75, 85, 86, 225-227, 246-248, 259-264, 280, 281, 295-297, 326, 352, 353 and 363 or any major part thereof. Each possibility is a separate embodiment.

According to some embodiments, the plant may be a rice plant and the polynucleotide may reduce the level of a target gene having any of the nucleotide sequences set forth in SEQ ID NO: 43, 44, 52-57, 73-75, 85, 86, 225-227, 246-248, 259-264 or any major part thereof. Each possibility is a separate embodiment.

According to some embodiments, the plant may be a rice plant and the polynucleotide may reduce the level of the protein having any of the amino acid sequences set forth in SEQ ID NO: 407-412, 416-425, 437-442, 449-467, 589-598, 610-615, 623-638, 644-649, 659-662, 690-695, 716-723, 727 and 728 or any major part thereof. Each possibility is a separate embodiment.

According to some embodiments, the plant may be a rice plant and the polynucleotide (i.e., the dsRNA) may have the nucleotide sequences set forth in SEQ ID NO: 742-747.

In certain embodiments, polynucleotide compositions and methods provided herein typically effect regulation or modulation (e. g., suppression) of gene expression during a period of the life of the treated plant of several days to several weeks or longer and typically in systemic fashion. For instance, within days of treating a plant leaf with a polynucleotide composition of this invention, primary and transitive siRNAs can be detected in other leaves lateral to and above the treated leaf and in apical tissue. In certain embodiments, methods of systemically suppressing expression of a gene in a plant, the methods comprising treating said plant with a composition comprising at least one polynucleotide and a transfer agent, wherein said polynucleotide comprises at least 18 or at least 19 contiguous nucleotides that are essentially identical or essentially complementary to a gene or a transcript encoding a target gene of the plant are provided, whereby expression of the gene in said plant or progeny thereof is systemically suppressed in comparison to a control plant that has not been treated with the composition.

Compositions used to suppress a target gene can comprise one or more polynucleotides that are essentially identical or essentially complementary to multiple genes, or to multiple segments of one or more genes. In certain embodiments, compositions used to suppress a target gene can comprise one or more polynucleotides that are essentially identical or essentially complementary to multiple consecutive segments of a target gene, multiple non-consecutive segments of a target gene, multiple alleles of a target gene, or multiple target genes from one or more species.

In certain embodiments, the polynucleotide includes two or more copies of a nucleotide sequence (of 18 or more nucleotides) where the copies are arranged in tandem fashion. In another embodiment, the polynucleotide includes two or more copies of a nucleotide sequence (of 18 or more nucleotides) where the copies are arranged in inverted repeat fashion (forming an at least partially self-complementary strand). The polynucleotide can include both tandem and inverted-repeat copies. Whether arranged in tandem or inverted repeat fashion, each copy can be directly contiguous to the next, or pairs of copies can be separated by an optional spacer of one or more nucleotides. The optional spacer can be unrelated sequence.

While there is no upper limit on the concentrations and dosages of polynucleotide molecules that can be useful in the methods and compositions provided herein, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, leaves, roots, or seeds.

Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e. g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet® L-77 organosilicone preparation is used as a pre-spray treatment of plant leaves or other plant surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet® L-77 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

According to some embodiments, the polynucleotide compositions that comprise an organosilicone preparation can comprise a salt such as ammonium chloride, tetrabutylphosphonium bromide, and/or ammonium sulfate. Ammonium chloride, tetrabutylphosphonium bromide, and/or ammonium sulfate can be provided in the polynucleotide composition at a concentration of about 0.01% to about 5% (w/v).

According to some embodiments, other useful transfer agents or adjuvants to transfer agents that can be used in polynucleotide compositions provided herein include surfactants and/or effective molecules contained therein. Surfactants and/or effective molecules contained therein include, but are not limited to, sodium or lithium salts of fatty acids (such as tallow or tallowamines or phospholipids) and organosilicone surfactants. In certain embodiments, the polynucleotide compositions that comprise a transfer agent are formulated with counter-ions or other molecules that are known to associate with nucleic acid molecules. Illustrative examples include, tetraalkyl ammonium ions, trialkyl ammonium ions, sulfonium ions, lithium ions, and polyamines such as spermine, spermidine, or putrescine.

In certain embodiments, the polynucleotide compositions further include glycerin. Glycerin can be provided in the composition at a concentration of about 0.1% to about 1% (w/v or v/v). A glycerin concentration of about 0.4% to about 0.6%, or about 0.5% (w/v or v/v) can also be used in the polynucleotide compositions that comprise a transfer agent.

In certain embodiments, the polynucleotide compositions further include an organic solvent. Non-limiting examples of suitable organic solvents include, but are not limited to, DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions).

In certain embodiments, the polynucleotide compositions further include a naturally derived or synthetic oil with or without a surfactant and/or an emulsifier. Non-limiting examples of suitable oils include, but are not limited to, plant-sourced oils, crop oils, paraffinic oils, polyol fatty acid esters, or oils with short-chain molecules modified with amides or polyamines such as polyethyleneimine or N-pyrrolidine.

Compositions and methods of the invention are useful for modulating or suppressing the expression of an endogenous target gene or transgenic target gene in a plant cell or plant. In certain embodiments of the methods and compositions provided herein, expression of genes targeted by the polynucleotides disclosed herein can be suppressed completely, partially and/or transiently to result in improved yield.

Target genes and plants containing those target genes can be obtained from: i) row crop plants; ii) vegetable plants; iii) culinary plants; iv) fruit plants; v) a tree grown for ornamental or commercial use; or, vi) trees in natural forests or, vii) an ornamental plant. The methods and compositions provided herein can also be applied to plants produced by a cutting, cloning, or grafting process.

Compositions comprising a polynucleotide and a transfer agent provided herein can be topically applied to a plant or plant part by any convenient method, e.g., spraying or coating with a powder, or with a liquid composition comprising any of an emulsion, suspension, or solution. Such topically applied sprays or coatings can be of either all or of any a portion of the surface of the plant or plant part. Similarly, the compositions comprising a transfer agent or other pre-treatment can in certain embodiments be applied to the plant or plant part by any convenient method, e. g., spraying or wiping a solution, emulsion, or suspension. Compositions comprising a polynucleotide and a transfer agent provided herein can be topically applied to plant parts that include, but are not limited to, roots, flowers, stems, tubers, meristems, ovules, fruit, anthers, pollen, leaves, or seeds.

According to some embodiments, the composition may be provided by irrigation e.g. using an existent or a designated irrigation system.

Application of compositions comprising a polynucleotide and a transfer agent to seeds is specifically provided herein. Seeds can be contacted with such compositions by spraying, misting, immersion, and the like. According to some embodiments, progeny plants, plant parts, derived from treated seeds will exhibit an improved yield that result from suppressing expression of the target gene.

Various methods of spraying compositions on plants or plant parts can be used to topically apply to a plant surface a composition comprising a polynucleotide that comprises a transfer agent. In the field, a composition can be applied with a boom that extends over the crops and delivers the composition to the surface of the plants or with a boomless sprayer that distributes a composition across a wide area. Agricultural sprayers adapted for directional, broadcast, or banded spraying can also be used in certain embodiments. Sprayers adapted for spraying particular parts of plants including, but not limited to, leaves, the undersides of leaves, flowers, stems, male reproductive organs such as tassels, meristems, pollen, ovules, and the like can also be used. Compositions can also be delivered aerially, such as by a crop-dusting airplane. In certain embodiments, the spray can be delivered with a pressurized backpack sprayer calibrated to deliver the appropriate rate of the composition.

In certain embodiments, plant parts can be sprayed either pre- or post-harvest to provide improved yield in the plant part. Compositions can be topically applied to plant parts attached to a plant by a spray as previously described. Compositions can be topically applied to plant parts that are detached from a plant by a spray as previously described or by an alternative method. Alternative methods for applying compositions to detached parts include, but are not limited to, passing the plant parts through a spray by a conveyor belt or trough, or immersing the plant parts in the composition.

Compositions comprising polynucleotides and transfer agents can be applied to plants or plant parts at one or more developmental stages as desired and/or as needed. Application of compositions to pre-germination seeds and/or to post-germination seedlings is provided in certain embodiments. Seeds can be treated with polynucleotide compositions provided herein by methods including, but not limited to, spraying, immersion, or any process that provides for coating, imbibition, and/or uptake of the polynucleotide composition by the seed. Seeds can be treated with polynucleotide compositions using seed batch treatment systems or continuous flow treatment systems. Seed treatment can also be applied in laboratory or commercial scale treatment equipment such as a tumbler, a mixer, or a pan granulator. A polynucleotide composition used to treat seeds can contain one or more other desirable components including, but not limited to liquid diluents, binders to serve as a matrix for the polynucleotide, fillers for protecting the seeds during stress conditions, and plasticizers to improve flexibility, adhesion and/or spreadability of the coating. In addition, for oily polynucleotide compositions containing little or no filler, drying agents such as calcium carbonate, kaolin or bentonite clay, perlite, diatomaceous earth or any other adsorbent material can be added.

Application of the compositions in early, mid-, and late vegetative stages of plant development is provided in certain embodiments. Application of the compositions in early, mid-, and late reproductive stages is also provided in certain embodiments. Application of the compositions to plant parts at different stages of maturation is also provided.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1—Verifying Improved Yield Trait of Plants

The applying of the composition (by irrigation or spraying for example) is timed according to the transcription of the target gene and the desired trait it affects. Examples of suitable timing is outlined in TABLE 2, which shows selected examples of suitable timing for applying compositions based on the genes targeted (here for oilseed rape) and indication of same. The dsRNA mixture is applied according to the timing of the expression of the specific gene, namely a week before, on the expected peak of expression and a week after the peak of its expression. Following the treatment, the phenotype of the plant, with regards to the yield trait targeted, is inspected, as for example outlined in TABLE 2 which shows selected examples of traits and their related genes.

### Example 2—Testing Transfer Agent Permeability

In order to test the permeability of a transfer agent a 0.01% to 1% or 0.1-10 mg/ml transfer agent solution was sprayed on a leaf and the contact angle evaluated using standard methods.

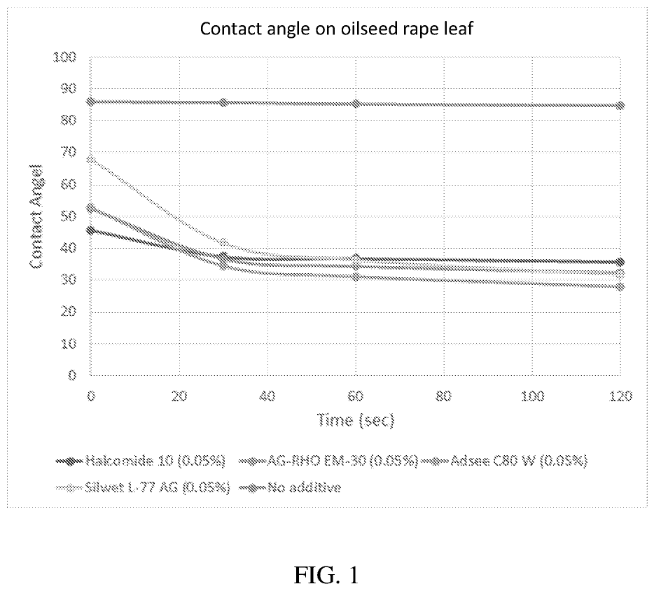

The efficiency of the tested transfer agents is shown in FIG. 1 which shows the contact angle (indicative of penetration) as a function of time after application on a leaf of the plant. As seen, all the tested transfer agents significantly reduced the contact angle as compared to when no transfer agent was applied.

### Example 3—Increased Branching by Targeting BRC1 in an Oilseed Rape

dsRNA molecules (SEQ ID NO: 730) directed against BnBRC1 of oilseed rape (Brassica napus) (SEQ ID NO: 1) was applied using a sprayer at a dsRNA concentration of 1 ng/ml to 1 mg/ml diluted in 0.01% to 1% or 0.1-10 mg/ml surfactant, here Siloxane Polyalkyleneoxide Copolymer (Silwet® L-77 AG) however other surfactants such as those listed in TABLE 1 may likewise be used. The BnBRC1 (SEQ ID NO: 730) dsRNA was applied from beginning of bolting to second inflorescence appearance.

Following the treatment, the branching of the plant was evaluated by visual inspection. As shown in FIG. 3, which shows illustrative images of Brassica napus plants following topical application of the dsRNA, the branching of the plant significantly increased (5-6 branches per plant in mock treated control plants (left) and 9-10 branches per plant in BnBRC1 dsRNA treated plants with a dsRNA comprising the sequence set forth in SEQ ID NO: 730 (right).

### Example 4—Ectopic Application of dsRNA Affecting Petal Configuration of Oilseed Rape in Greenhouse and Field

dsRNA molecules (SEQ ID NO: 733) directed against Brassica napus gene BnPTL (SEQ ID NO: 286) was applied using a hand sprayer at a dsRNA concentration of 1 ng/ml to diluted in 0.01% to 1% or 0.1-10 mg/ml surfactant, here Siloxane Polyalkyleneoxide Copolymer (Silwet® L-77 AG), however other surfactants such as those listed in TABLE 1 may likewise be used. The dsRNA was applied when the plant reached 70% to full stem length.

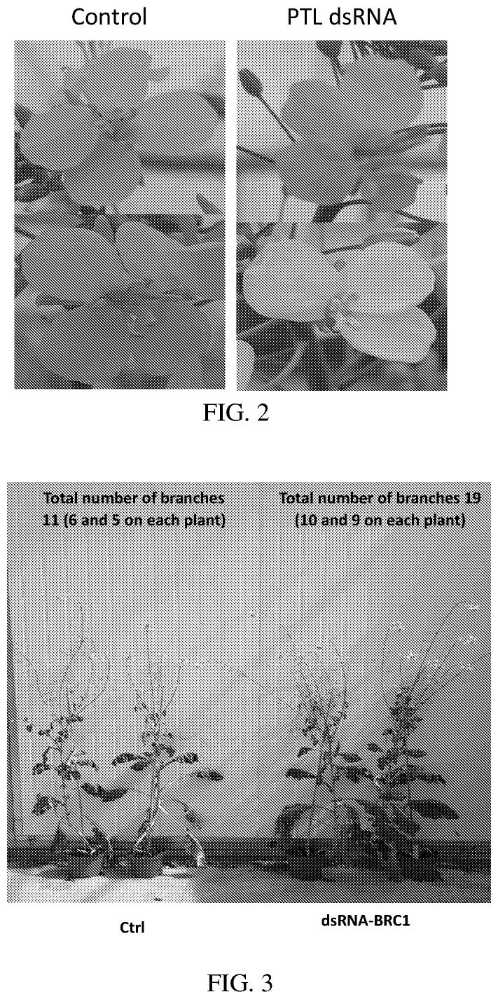

Following the treatment, the number of petals was evaluated by visual inspection. FIG. 2, shows preliminary, illustrative images of oilseed rape plants, here Brassica napus following topical application of the BnPTL dsRNA. In mock treated plants (left) normal petals configuration is seen, while the BnPTL dsRNA treated plant (right) showed abnormal petals configuration (three petals per flower or asymmetrical flowers), demonstrating the ability of the topically applied dsRNA to affect the petal configuration of oilseed rape plants.

### Example 5—Ectopic Application of dsRNA for Yield Enhancement—Oil Rapeseed

Oilseed rape var. Belinda seeds (Brassica napus) were sown in 3-liter pots in a controlled greenhouse, one plant per pot, watered and fertilized once a day. The plants were treated with dsRNA molecules (SEQ ID NO: 733) directed against BOOT: gene (SEQ ID NO: 286)—hereinafter referred to a treatment ‘A’ in all further descriptions) and dsRNA molecules (SEQ ID NO: 730) directed against BnBRC1 gene (SEQ ID NO: 1)—hereinafter referred to a treatment ‘B’. The treatment was applied according to the appropriate development stage of the plant, as set forth in TABLE 2 below, in a concentration of 1 and 10 μg/ml dsRNA diluted in surfactant (Silwet® L-77 AG), 10 ml per plant.

Phenotypic evaluations were made one month after applying the treatment and the result compared to Ctrl (surfactant only), for flower morphology of ‘A’ (BnPTL dsRNA) and branch number for ‘B’ (BnBRC1 dsRNA).

### Method

Oilseed rape seeds were sown in 2×1,000 m² fields, in the Sharon region, Israel (32° 10′1.55″N 34° 52′33.96″E), divided into 12 rows of 52 m×0.8 m, each row spaced by a 40 cm clear lane.

The seeds were sown in about 15 cm distance between each seed, and in 2 cm depth using hand pushed sower.

Each row was plotted to 0.8 m×2 m divided by 1 m of untreated plants as spacers between adjacent different treatments, about 70 plants per plot.

Five dsRNAs were tested, namely:
1) dsRNA molecules (SEQ ID NO: 733) directed against Brassica napus BOIL gene (SEQ ID NO: 286)—hereinafter termed treatment ‘A’,
2) dsRNA molecules (SEQ ID NO: 730) directed against Brassica napus BnBRC1 gene (SEQ ID NO: 1)—hereinafter termed treatment ‘B’,
3) dsRNA molecules (SEQ ID NO: 731) directed against Brassica napus BnCKX2 gene (SEQ ID NO: 158) hereinafter termed treatment ‘C’;
4) dsRNA molecules (SEQ ID NO: 732) directed against Brassica napus BnKIN10 gene (SEQ ID NO: 62)—hereinafter termed treatment ‘D’;
5) dsRNA molecules (SEQ ID NO: 729) directed against Brassica napus BnADPG1 gene (SEQ. ID NO: 235)—hereinafter termed treatment ‘E’.

The dsRNAs were sprayed with 200 ml of water supplemented with dsRNA and surfactant for each plot. The dsRNA treatment was applied according to the peak of expected expression timing of each selected gene during the plants' growing stage (as set forth in TABLE 2).

TABLE 2
Oilseed rape plant's development stage used for dsRNA
application for each gene and their phenotype evaluations.

| | Desired | Target | |
|---|---|---|---|
| Crop | Trait | Gene Name | Target Gene Annotation |
| Oilseed | Increased | BnBRC1 | Protein Transcription factor |
| Rape | Branching | | TCP18 BRANCHED1 (BRC1) |
| | | BnBRC1L | BRC1 Like |
| | | BnMAX1-4 | Cytochrome P450 |
| | Increased | BnICK1-7 | Cyclin-Dependent Kinase |
| | Seed Size | | (CDK) Inhibitor 1-7 |
| | | BnCKX1-7 | Cytokinin Oxidase 1-7 |
| | | BnGA2ox1-8 | gibberellin 2-oxidase 1 |
| | | BnDA1 | Ubiquitin receptor |
| | | BnDA2 | E3 ligase |
| | | BnEOD1 | E3 ligase |
| | | BnAP2 | APETALA 2 (AP2) |
| | | | Transcription factor |
| | Increased | BnSAMBA.1 | plant-specific anaphase- |
| | Seed | | promoting complex/cyclosome |
| | Number | | (APC/C) regulator |
| | Large | BnMYB12 | MYB transcription factor 12 |
| | Siliques | BnMYB111 | MYB transcription factor 111 |
| | | BnCHI | chalcone isomerase |
| | | BnF3H | naringenin,2-oxoglutarate 3- |
| | | | dioxygenase |
| | | BnF3′H | flavonoid 3′-monooxygenase |
| | | BnFLS | flavonol synthase 3-like |
| | | BnMIM858 | miR858 target mimic |
| | Decreased | BnNST1 | NAC SECONDARY WALL |
| | Shattering | | THICKENING PROMOTING |
| | | | FACOTR1 |
| | | BnSND1 | SECONDARY WALL- |
| | | | ASSOCIATED NAC DOMAIN |
| | | | PROTEIN 1 |
| | | BnADPG1 | ARABIDOPSIS |
| | | | DEHISCENCE ZONE |
| | | | POLYGALACTURONASE 1 |
| | | BnADPG2 | ARABIDOPSIS |
| | | | DEHISCENCE ZONE |
| | | | POLYGALACTURONASE 2 |
| | Petals | BnPTL | trihelix transcription factor |
| | reduction | | PTL-like |
| | Early | BnARP6 | ACTIN-RELATED |
| | Flowering | | PROTEIN 6 |
| | | BnFLC1-5 | MADS-box protein |
| | | | FLOWERING LOCUS C 1-5 |
| | | BnSVP | SHORT VEGETATIVE |
| | | | PHASE (SVP) |
| | Late | BnPIF4 | PHYTOCHROME |
| | flowering | | INTERACTING FACTOR 4 |
| | | | (PIF4) |
| Soy | Increased | GmBRC1 | TCP transcription factor |
| | branching | | BRANCHED1 (BRC1) |
| | | GmFRZ2 | Rice homologue of Cdh1. |
| | | | activator of the anaphase |
| | | | promoting complex/cyclosome |
| | | | (APC/C) complex |
| | Seed | GmBS1 | Plant-specific transcription |
| | Filling | | regulator |
| | | GmPLDα1 | Phospholipase D alpha 1 |
| | | | (PLD alpha 1). Positive regulator |
| | | | of abscisic acid (ABA) mediated |
| | | | stomatal movements. |
| | | GmJAG1 | Putative zinc finger |
| | | | transcription factor |
| | Increased | GmCKX1 | Cytokinin Oxidasee 1 |
| | Seed | | (CKX1) |
| | Number | | |
| Rice | Incresed | OsBRC1 | TCP transcription factor |
| | Tillering | | BRANCHED1 (BRC1) |
| | | OsGSK2 | SHAGGY-like kinase |
| | Increased | OsPIN5b | Auxin efflux carrier-like |
| | Panicles | | protein |
| | | OsFZP | Ethylene-responsive |
| | | | transcription factor |
| | Grain | OsSKIN1 | plant-specific SnRK1A- |
| | Filling | | interacting negative regulator |
| | | OsGNI1 | homeodomain leucine zipper |
| | | | class I (HD-Zip I) transcription |
| | | | factor |

For each treatment, two dsRNA dosages (1 or 10 μg/ml) and two spray regimens (1 or 5 times in the 1^{st} field and 1 or 3 times in the 2^{nd} field) were conducted, as set forth in TABLE 3. Gaps between subsequent treatments is one week.

TABLE 3
Field trials parameters.

| | Surfactant | Number of treatments |
|---|---|---|
| Field | Silwet ® L-77 AG | 1 - on the timing as in |
| 1 | (0.03%) | TABLE 2 |
| | | 5 - two weeks before, one |
| | | week before, on the timing as TABLE 2, |
| | | one week after and two weeks after the |
| | | timing as TABLE 2 |
| Field | GENAGEN ™ 4166 | 1 - on the timing as in |
| 2 | (0.05%) | TABLE 2 |
| | | 3 - one week before, on |
| | | the timing as TABLE 2 and one week |
| | | after the timing as in TABLE 2 |

Surfactant only (without dsRNA) in the same timing was utilized as control (ctrl).

Each treatment was repeated 10 times in different plots, the sprayings were performed using “Solo” 2-liter hand sprayer with the smallest drop size.

At the end of the growing season the plots were hand harvested, dried for one week, processed by a threshing machine (Classic ST, Wintersteiger, Germany), and weighted for net seed weight for each plot/treatment.

All field data was statistically analyzed (P<0.1) for each dsRNA treatment compared to its relevant Ctrl.

The weight of 1,000 seeds was measured using a designated seed counting machine (Contador, Pfeuffer, Germany) with 5 replicates for each plot, combined with total weight of fixed volume. Oil content were measured according to Soxhlet” extraction method at the “Biotechnology Engineering Faculty, Ben-Gurion University, Be'er Sheva, Israel, using hexane as a solvent.

### Results

### Treatment a

### Flower Morphology

For treatment ‘A’ flower morphology was evaluated three weeks after application of the sdRNA.

As seen from FIG. 2, a change in flower morphology was observed in the dsRNA treated plants only. In each inflorescence at least one flower was lacking one petal, as compared to controls. In addition, inflorescence development and flowering were delayed by about 2 weeks in the dsRNA treated plants, as compared to control.

### Light Penetration Percentage:

Three weeks after spray application of the dsRNA (SEQ ID NO: 733), light penetration percentage at the base of the inflorescences was measured. The measurements took place after flowering peak. dsRNA treatment caused a significant reduction in light penetration for all treatments applied, namely: 1-1 (1-1=1 μg/ml, 1 treatment), 10-1 (10-1=10 μg/ml, 1 treatment), 1-5 (1-5=5 treatments) and 10-5 (10-5=10 μg/ml, 5 treatments) causing 42.3%, 45.1%, 47.6% and 50% reduction in light penetration respectively, as compared to controls.

These results show that reducing BnPTL expression (SEQ ID NO: 286) by ectopic administration of dsRNA molecules targeting PTL reducing petals number which in turn will lead to increase light penetration to the plant's lower parts and will increased total photosynthesis efficiency and yield.

### Seed Weight:

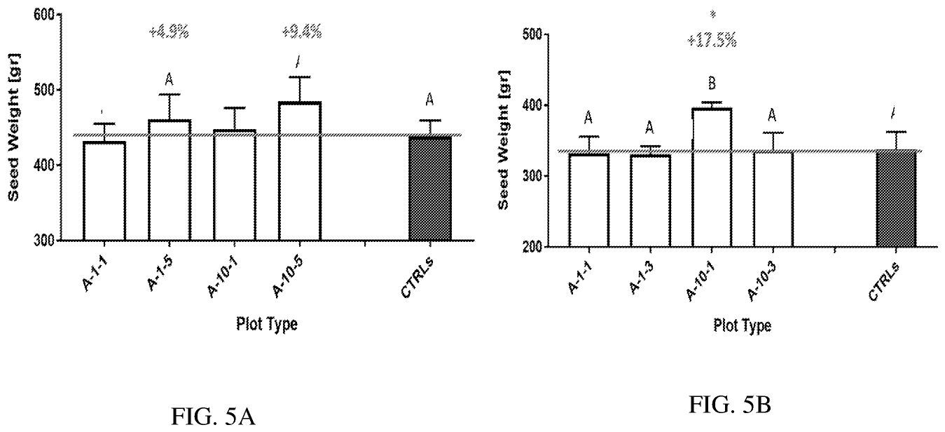

In the 1^{st} field, treatments 1-5 and 10-5 increased seed weight by 4.9% and 9.4% respectively (FIG. 5A). In the 2^{nd} field, treatment 10-1 increased seed weight by 17.5%. (FIG. 5B).

These results indicate that reducing BnPTL expression by ectopic administration of dsRNA targeting BnPTL can increase seed weight.

### Oil Content:

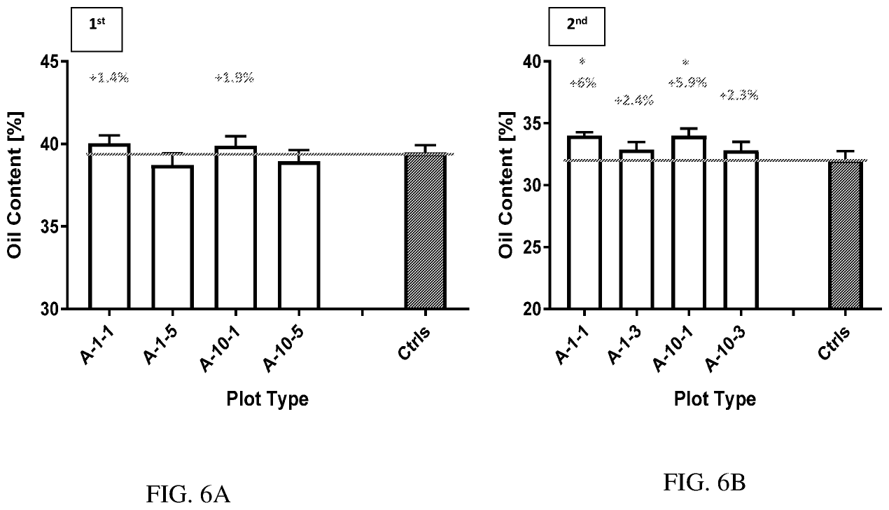

In the 1^{st} field a consistent increase in oil content was observed in the dsRNA treated plants 1.4%, 1.9% and 1.1% (for treatments 1-1, 1-5 and 10-1, respectively—FIG. 6A).

In the 2^{nd} field, a larger increase of 6%, 2.4%, 5.9% and 2.3% (for treatments 1-1, 1-3, 10-1 and 10-3, respectively—FIG. 6B) was observed.

These results indicate that reducing BnPTL expression by ectopic administration of dsRNA targeting BnPTL can increase oil content of the oilseed rape plant.

### Treatment B

### Branch Numbers:

For treatment ‘B’ the number of branches was evaluated three weeks after application of the dsRNA.

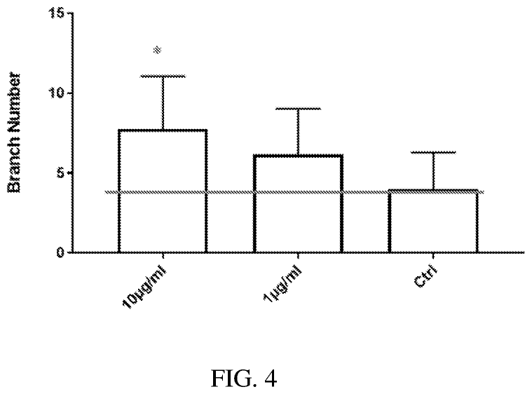

As seen from FIG. 3, the number of branches increased, as a result of the dsRNA treatment (10 μg/ml) and as further seen from FIG. 4 the increase in the number of branches was dose-dependent. (P<0.1).

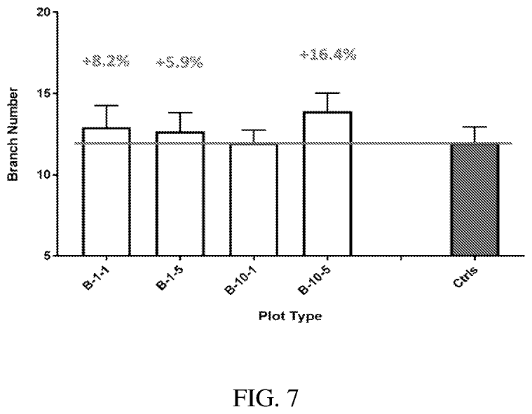

As seen from FIG. 7, showing field results of targeting BnBRC1 (SEQ ID NO: 1) expression by ectopic administration of dsRNA (SEQ ED NO: 730 targeting BnBRC1) resulted in an increase in the number of branches) 8.2%, 5.9% and 16.4% for treatments 1-1, 1-5 and 10-5 respectively as compared to controls.

### Treatment C

### Seed Weight:

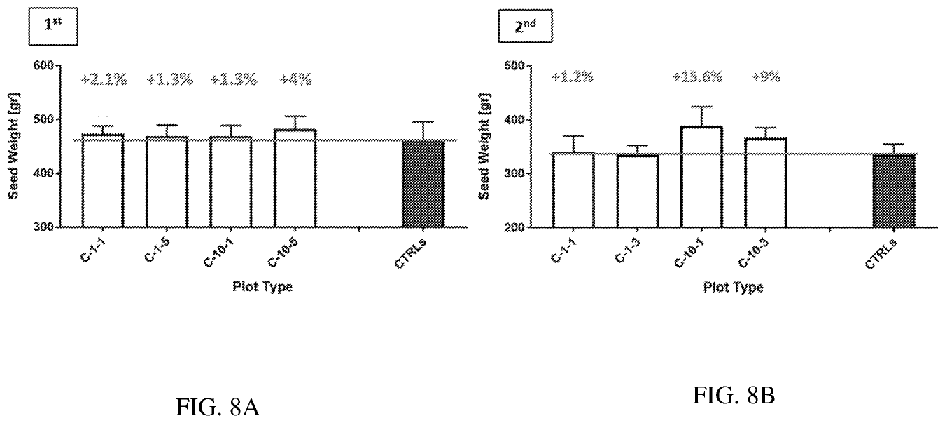

As seen from FIGS. 8A and 8B, targeting BnCKX2 (SEQ ID NO: 158) expression by ectopic administration of dsRNA (SEQ ID NO: 731) targeting CKX2 resulted in an increased seed weight in both fields tested. In the 1^{st} field, treatments 1-1, 1-5, 10-1 and 10-5, the increase was 2.1%, 1.3%, 1.3% and 4% respectively (FIG. 8A). In the 2^{nd} field treatments 1-1, 10-1 and 10-3 increased seed weight by 1.2%, 15.6% and 9%, respectively (FIG. 8B).

### 1,000 Seed Weight and Seed Size:

In addition, targeting BnCKX2 expression by ectopic administration of dsRNAs targeting BnCKX2 resulted in an increased 1,000 seed weight by 3.9%, 3.5% and 1.6% for treatments 1-1, 1-5 and 10-1 of the 1^{st} field, relative to Ctrl. The seed size in treatments 1-1, 1-5 and 10-5 likewise increased by 1.1%, 5.1% and 1.2% respectively.

In the 2^{nd} field, targeting BnCKX2 caused an increase to the 1,000 seed weight by 5.8%, 1.5% and 1.1% for treatments 1-3, 10-1 and 10-3, respectively. The seed size, in treatments 1-3, 10-1 and 10-3 was likewise increased by 4.1%, 0.7% and 3.8% respectively.

These results clearly show that targeting BnCKX2 expression by ectopic administration of dsRNAs targeting BnCKX2 increases weight and size of oilseed rape seeds.

### Treatment E

### Seed Weight:

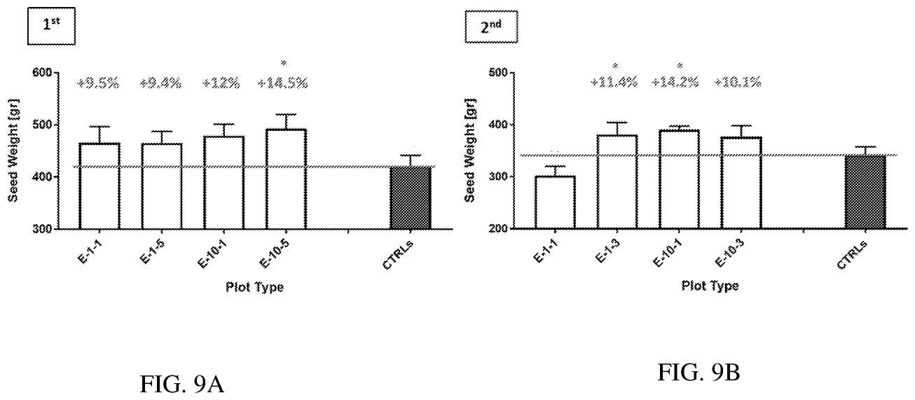

As seen in FIGS. 9A and 9B, targeting BnADPG1 (SEQ ID NO: 235) expression by ectopic administration of dsRNA (SEQ ID NO: 729) targeting BnADPG1 resulted in an increased seed weight in both fields tested. In the 1^{st} field, treatments 1-1, 1-5 and 10-1, the increase was 9.5%, 9.4%, 12% respectively. In the 2^{nd} field treatments 1-3, 10-1 and 10-3 increased seed weight by 11.4%, 14.2% and 10.1%, respectively.

### 1,000 Seed Weight and Seed Size:

Targeting ADPG1 expression by ectopic administration of dsRNAs targeting BnADPG1 resulted in an increased 1,000 seed weight by 4.5%, 7.7%, 6% and 7% for treatments 1-1, 1-5, 10-1 and 10-5 of the 1^{st} field, relative to Ctrl. The seed size in treatments 1-1, 1-5, 10-1 and 10-5 was likewise increased by 6.8%, 8.2%, 1.1% and 8.1% respectively.

In the 2^{nd} field, targeting BnADPG1 caused an increase to the 1,000 seed weight by 14.7%, 3% and 13.3% for treatments 1-3, 10-1 and 10-3, respectively. The seed size, in treatments 1-1, 1-3, 10-1 and 10-3 was likewise increased by 2%, 13.3%, 2.2% and 9.6% respectively.

These results clearly show that targeting BnADPG1 expression by ectopic administration of dsRNAs targeting BnADPG can increase weight and size of oilseed rape seeds.

### Oil Content:

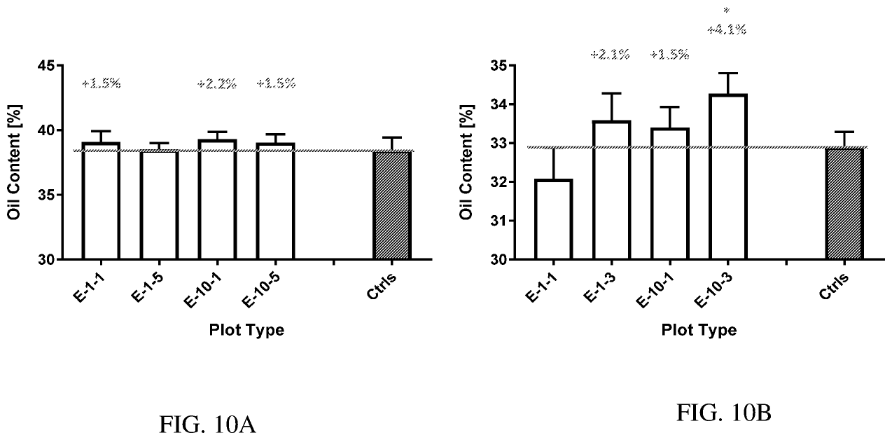

In the 1^{st} field an increase in oil content was observed in the BnADPG1-dsRNA treated plants (1.5%, 2.2% and 1.5% for treatments 1-1, 10-1 and 10-5, respectively—FIG. 10A). Similarly, In the 2^{nd} an increase in oil content of 2.1%, 1.5% and 4.1% was observed in treatments 1-3, 10-1 and 10-3 respectively, as compared to control (FIG. 10B).

### Example 6—Ectopic Application of dsRNA for Yield Enhancement—Rice (Oryza sativa)

### Materials and Methods:

Rice (Oryza sativa spp.) seeds were sown in 3-liter pots, one seed per pot in greenhouse, watered and fertilized once a day.

The plants were treated as with 10 μg/ml (in water and surfactant) of the below listed dsRNAs:
1) dsRNA molecules (SEQ. ID NO: 742) directed against OsBRC1 of rice (SEQ ID NO: 43),
2) dsRNA molecules (SEQ ID NO: 744) directed against OsPIN5b of rice (SEQ ID NO: 86),
3) dsRNA molecules (SEQ ID NO: 746) directed against OsSKIN1 of rice (SEQ ID NO: 52),

The dsRNAs were applied according to the plants' development stage, as set forth in TABLE 4 below.

TABLE 4
Rice (O. sativa) plants developing stage
used for specific dsRNA treatments.

| dsRNA | Development stage of treatment |
|---|---|
| SEQ ID NO: 742 | axillary buds start to emerge |
| SEQ ID NO: 744 | Plants at maximum tiller number |
| SEQ ID NO: 746 | Plants at seed filling stage |

The dsRNAs were applied as one time spraying, in a concentration of either 1 or 10 μg/ml, diluted in surfactant (Silwet® L-77 AG), 10 ml per plant, six pots/plants for each treatment.

The treatments were evaluated one month after application of dsRNA and compared to Ctrl. For OsBRC1 the number of axillary tillers was evaluated, for OsPIN5b, the number of panicles was evaluated and for OsSKIN1 seed size was evaluated. Total seed weight was measured for all treatments.

### Results

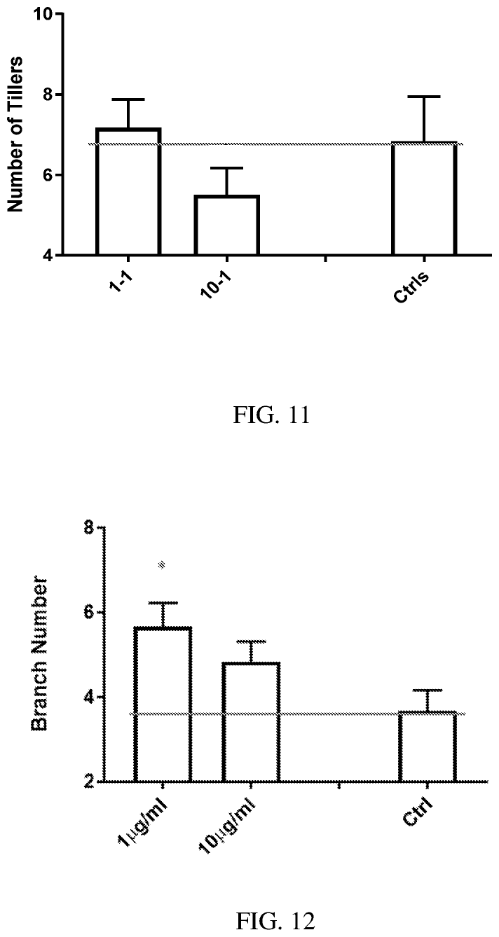

One month after treatment, total number of tillers of the plants treated with a dsRNAs targeting OsBRC1 of the rice plants (SEQ ID NO: 742) was counted and compared to control. Interestingly, treating the plants with a low concentration of OsBRC1-dsRNA caused a slight increase in the number of tillers, whereas a high concentration caused a decrease in the number of tillers (FIG. 11). This indicates that dsRNAs targeting OsBRC1 can be used to control the number of tillers in rice.

Three months after treatment, the number of panicles in the rice plants treated with dsRNAs targeting OsPIN5b (SEQ ID NO: 744 and 745) as comparted to control is evaluated.

Three months after treatment, the total seed weight is measured for all treatments.

### Example 7—Ectopic Application of dsRNA for Yield Enhancement—Soybean (Glycine max)

### Materials and Methods:

Soybean (Glycine max, Williams82 variety) seeds were sown in 3-liter pots, one seed per pot, in greenhouse, watered and fertilized once a day, or as needed.

Several examples of dsRNAs applications (as specified below), targeted to cause a phenotypic change and increase yield were tested:
1) SEQ ID NO: 734 directed against GmBRC1 of soybean (SEQ ID NO: 15).
2) SEQ NO: 738 directed against GmJAG1 of soybean (SEQ ID NO: 153).
3) SEQ ID NO: 736 directed against GmBS1 of soybean (SEQ ID NO: 116).
4) SEQ ID NO: 740 directed against GmPLDα1 of soybean (SEQ ID NO: 124).

The dsRNAs were applied according to the plants' development stage, as set forth in TABLE 5 below.

TABLE 5
Soybean (G. max) plants developing stage
used for specific dsRNA treatments.

| dsRNA | Development stage of treatment |
|---|---|
| SEQ ID NO: 734 | Bolting stage |
| SEQ ID NO: 738 | Flowering stage |
| SEQ ID NO: 736 | Seed filling stage |
| SEQ ID NO: 740 | Seed filling stage |

The dsRNAs were applied as two sprays, 1^{st} spray at the presumed peak of the gene expression timing and 2^{nd} spray two weeks later. The dsRNA was applied in a concentration of 1 μg/ml or 10 μg/ml, diluted in surfactant (Silwet® L-77 AG), 10 ml per covering the whole plant surface, six pots/plants for each treatment. Each dsRNA trial was conducted in six replicas.

The treatments were evaluated one month after applying the dsRNA and compared to Ctrl. For GmBRC1 targeting, the number of axillary branches was evaluated; for GmJAG1 targeting, seed number per pod was evaluated, for GmBS1 targeting, the seed size was evaluated; and for GmPLDα1, seed weight/filling was evaluated. In addition, total seed weight was measured for all treatments.

### Results

One month after treatment plants treated with dsRNA targeting GmBRC1—were evaluated for total number axillary branches. As seen in FIG. 12 dsRNA treated plants had higher number of branches as compared to the Ctrl plants. Interestingly, the largest increase (54.1%) was observed for the lower dsRNA concentration.

One month after dsRNA treatment targeting GmJAG1, the plants are evaluated for total number of seeds per pod.

After total drying of the plants' pods, the plants treated with dsRNA targeting GmBS1 are evaluated for seed size, and the plants treated with dsRNA targeting GmPLDα1 for total seed weight.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A composition comprising:
a dsRNA molecule that comprises at least 18 contiguous nucleotides that are essentially identical or essentially complementary to a portion of a plant gene or a transcript of the plant gene; and
a transfer agent configured to facilitate permeation of the dsRNA molecule into cells of the plant;
wherein permeation of the dsRNA molecule into cells of the plant causes a transient reduction in the expression of the gene, and
wherein the transient reduction in the expression of the gene causes a change in a trait of the plant, the trait of the plant selected from the group consisting of increased branching, increased grain filling, increased T6P levels, increased number of panicles, increased seed filling, increased seed number, increased seed size, decreased shattering, decreased abscise tissue formation, increased number of tillers, increased heading in the plant, petal reduction, increase siliques size, late or early flowering, delayed senescence and any combination thereof.

2. The composition of claim 1, wherein the plant gene is selected from ADPG1, PTL, CKX2, BRC1, KIN10, SKIN1, PIN5b, JAG1, BS1, PLDα1 and/or or any homolog or combination thereof.

3. The composition of claim 1, wherein the plant gene is selected from ADPG1, PTL, CKX2, BRC1 and/or or any homolog or combination thereof.

4. The composition of claim 1, wherein the plant is an oilseed rape plant and wherein the dsRNA molecule comprises at least 18 contiguous nucleotides that are essentially identical or essentially complementary to a portion of a sequence encoding any of the amino acid sequences set forth in NO SEQ ID NO: 599, SEQ ID NO: 650, SEQ ID NO: 522 and SEQ ID NO: 365.

5. The composition of claim 1, wherein the plant is a soybean plant, and wherein the dsRNA molecule comprises at least 18 contiguous nucleotides that are essentially identical or essentially complementary to a portion of a sequence encoding any of the amino acid sequences set forth in SEQ ID NO: 379, SEQ ID NO: 603, SEQ ID NO: 655, SEQ ID NO: 564, SEQ ID NO: 517, SEQ ID NO: 480 and SEQ ID NO: 488.

6. The composition of claim 5, wherein the plant is a soybean plant, and wherein the dsRNA molecule comprises at least 18 contiguous nucleotides that are essentially identical or essentially complementary to a portion of a sequence encoding any of the amino acid sequences set forth in SEQ ID NO: 379, SEQ ID NO: 517, SEQ ID NO: 480 and SEQ ID NO: 488.

7. The composition of claim 1, wherein the plant is a rice plant, and wherein the dsRNA molecule comprises at least 18 contiguous nucleotides that are essentially identical or essentially complementary to a portion of a sequence encoding any of the amino acid sequences set forth in SEQ ID NO: 407, SEQ ID NO: 610, SEQ ID NO: 659, SEQ ID NO: 589, SEQ ID NO: 416 and SEQ ID NO: 450.

8. The composition of claim 7, wherein the plant is a rice plant, and wherein the dsRNA molecule comprises at least 18 contiguous nucleotides that are essentially identical or essentially complementary to a portion of a sequence encoding any of the amino acid sequences set forth in SEQ ID NO: 407, SEQ ID NO: 416 and SEQ ID NO: 450.

9. The composition of claim 1, wherein said dsRNA molecule is at least about 50 bases in length.

10. The composition of claim 9, wherein said dsRNA molecule is at least about 200 bases in length.

11. The composition of claim 1, wherein the transfer agent comprises N, N-dimethyl Decanamide, coco amidopropyldimethyamine, Siloxane Polyalkyleneoxide Copolymer, AG-RHO® EM-30, Dimethylamide of C8/C10 fatty acid, esterified copolymer of glycerol, trisiloxane ethoxylate or any combination thereof.

12. A method for topically applying to a plant surface the composition of claim 1.

13. The method of claim 12, wherein the applying comprises spraying the composition onto the surface of a plant.

14. The method of claim 13, wherein the composition is sprayed onto the surface of a plant with a boom that extends over a crop, a boomless sprayer, an agricultural sprayer, a crop-dusting airplane, a pressurized backpack sprayer, a track sprayer, or a laboratory sprayer/submerger.

15. The method of claim 12, wherein the applying comprises providing the composition through an irrigation system.

16. The method of claim 12, wherein the plant surface is the surface of one or more plant part selected from the group consisting of hypocotyl, cotyledon, leaf, flower, stem, tassel, meristem, pollen, ovule, and fruit.

17. The method of claim 12, further comprising timing the applying of the composition at a desired developmental stage of the plant.
